(19) United States
(12) Patent Application Publication
(10) Pub. No.: US 2002/0064877 A1
(43) Pub. Date: May 30, 2002

(54) Methods of producing and using a human microglial cell line

(76) Inventor: Seung U. Kim, Vancouver (CA)

Correspondence Address: David Prashker, P.C., P.O. Box 5387, Magnolia, MA, 01930, US

(21) Appl. No.: 09/855,468

(22) Filed: May 15, 2001

Related U.S. Application Data
(63) Continuation of application No. PCT/US00/18777, filed on Jul. 10, 2000.

(51) Int. Cl. C12N 15/867; C12N 5/08

(52) U.S. Cl. 435/456; 435/368

(57) ABSTRACT

An immortalized human cell line has the characteristics of human microglia. It expresses the CD 8 and CD11c antigens. The immortalized human cell line has at least three of the following attributes: CD11b (Mac1), CD68, HLA-ABC, HLA-DR, IL-1b, IL-6, IL-8, IL-12, IL-15, TGF-b, TNF-a, MIP-1a, MIP-1b, MCP-1, P2Y1R, P2Y2R. Also disclosed is a method of transforming human microglial cells into an immortalized cell line, a method of testing drugs for effects on human microglial cells and a method of treating individuals experiencing neurodegenerative disorders.

### TECHNICAL FIELD

This invention is in the field of genetic engineering; more specifically, human microglial cells have been immortalized with an oncogene to produce an immortalized human microglial cell line.

### BACKGROUND

Microglia lay a critical role as resident immunocompetent cells and phagocytic cells in the central nervous system (CNS) and serve as scavenger cells in the event of infection, inflammation, trauma, ischemia and neurodegeneration in the CNS. Activated microglia are observed in pathological lesions in neurodegenerative diseases and may be involved in initiation or progression of CNS pathology. Neurodegeneration is preceded or is concomitant with functional responses of microglia including cell proliferation and secretion of host of molecules including oxygen radicals. proteases and pro-inflammatory cytokines.

Microglia were first described in 1919 by del Rio Hortega in silver carbonate stained brain preparations at the light microscope level; they are a morphologically distinct type of cell with long and branched processes. On the basis of studies in the developing CNS using silver staining or electron microscopy, microglia have at various times been described as having a mesodermal, monocytic or ectodermal origin. It has now shown that monocytes, which arc capable of differentiating into macrophages, enter the brain and retina during embryonic development and there differentiate into microglia. For that reason, many cell surface antigens are shared by microglia and macrophages. The antigens shared by microglia and macrophages include CD11b (Mac-I, b2 integrins), CD11c (LeuMS), CD45 (leukocyte common antigen), CD64 (Fc y receptor), CD68 (macrophage antigen), complement type 3 receptor (CR3) and MHC class I and II antigens.

Recent studies have indicated that the activation of microglia precedes or is concomitant with neuronal and glial cell degeneration in a variety of neurological diseases, including Alzheimer's disease, Parkinson's disease, stroke, brain trauma, AIDS-dementia, and multiple sclerosis. Thus, microglia-mediated neurotoxicity appears to be critical in tissue damage and neuronal death during the initiation and progression of neurological disorders. Earlier studies on human microglia have been mostly in histological sections and more recently in primary cultures of fetal or adult brain origin.

The CHME-5 cell line was obtained from human fetal microglia by transfection with the large T antigen f simian virus 40 (Jannbi, N., et al. 1995, Neurosci. Lett. 195:105-108, 1995). The transformed cells had macrophage characteristics of adherence and intra-cytoplasmic non-specific esterase activity. The cells phagocytized zymosan particles, but the phagocytic activity was low. Interferon-y activated the cells to express class II major histocompatibility complex antigens. Also, the cells produced interleukin-6 spontaneously, and this production was further increased after interleukin-1 a stimulation. They expressed several macrophage antigens but not the monocytic markers CD 14, CD4. CD68/Ki-M6 and CD11 c. Thus, these cells are similar, but not identical, to human microglial cells.

Studies of microglia and their roles in pathology have had serious limitations because it is difficult to obtain sufficient amounts of human microglia to study the detailed cellular and molecular characteristics of these cells. What is needed is a stable, greater, dependable source of microglial cells that more closely reflects the attributes of human microglial cells and does not rely on human fetal sources.

### BRIEF DESCRIPTION OF FIGURE

FIGS. 1A and 1B are phase-contrast photomicrographs of live human microglia isolated from a human embryonic brain (FIG. 1A) and of HM06.AI human microglial cells (FIG. 1B).

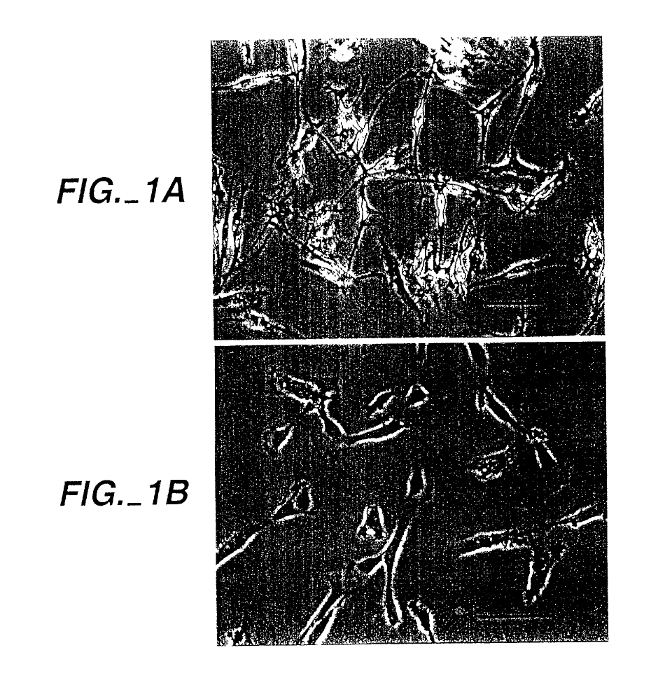

SUMMARY OF INVENTION

It is an object of this invention to provide an immortalized human cell line, which closely mirrors the characteristics of human microglia. It is also an object of this invention to test drugs and to treat neurodegenerative disorders.

Disclosed herein is an immortalized human microglial cell line expressing the CD68 and CD11c antigens. In another embodiment, the immortalized human microglial cell line has at least three of the following attributes: CD11b (Mac1), CD68, HLA-ABC, HLA-DR, IL-1b, IL-6, IL-8, IL-2, IL-12, TGF-b, TNF-a, MIP-Ia, MIP-1b, MCP-1, P2Y1R, P2Y2R.

Also disclosed is a method of transforming human microglial cells into an immortalized cell line by obtaining human microglial cells, culturing human microglial cells, transfecting the human microglial cells with arnphotropic replication-incompetent retroviral vector encoding v-rnyc oncogene transcribed from mouse leukemia virus LTR plus neomycin-resistant gene transcribed from an internal SV40 early promoter, and expanding the transfectants.

In another embodiment, there is provided a method of testing drugs for effects on human microglial cells, by providing immortalized human microglial cells, exposing the cells to the drug for a sufficient time to permit a reaction, and observing the reaction of the cells.

In a further embodiment, there is provided a method of treating an individual experiencing a neurodegenerative disorder, by administering a sufficient quantity of the immortalized human microglial cells to the individual with the neurodegenerative disorder. This neurodegenerative disorder preferably involves the individual's microglia cells. Preferably, the method treats the neurodegeneration disorder of Parkinson's Disease, Alzheimer's disease, ischemia, spinal cord damage, ataxia, or alcoholism.

### DETAILED DESCRIPTION OF INVENTION

We have recently produced and cloned several continuous cell lines of human microglia, HMO6 cell lines, by transfecting embryonic human microglia with a retroviral vector containing the cDNA encoding the v-myc oncogene.

There are many uses for an immortalized cell line of human microglia. The cells can be used to isolate neurotoxic or neurotrophic molecules naturally produced by human microglia or produced in response to inflammatory factors. Because microglia have been implicated in neurological disorders, such as Alzheimer's disease, Parkinson's disease, AIDS-dementia, ALS and MS, the microglia activated therein can be used in drug discovery of new drugs to treat the aforementioned conditions and inflammation.

Definitions:

“Human microglial cell line”, as used herein, means a human-derived cell line with microglial characteristics, including at least the following antigens CD68 and CD11c.

As used herein, “non-fetal” refers to the fact that the cells are expanded from immortalized cells, and there is no need to return to a fetal source.

It should be understood that a number of different vectors besides the described retroviral vector could be used to transform human microglia cells. The transformation of such cells is within the art, and cells transformed by any other vector can be tested as disclosed below to assure the presence of microglial phenotypic markers. Further, the invention is not limited to immortalizing cells with the oncogene v-myc. Many other oncogenes are known in the art and could be suitable for immortalizing microglia, pending testing as describe below.

Human immortalized microglial cells can be further genetically manipulated to produce additional proteins and/or drugs or prodrugs. Such proteins include but are not limited to chemokines, cytokines, and various marker proteins (e.g. LacZ and GFP). Such cells can be further genetically modified to block the production of proteins that may be overproduced by nervous system pathology. Upstream from the v-myc, there can be an activatable suppressor gene. Alternately, for human treatment, there can be a suicide gene.

General Methods

Standard molecular biology techniques known in the art and not specifically described are generally followed as in Sambrook et al., MOLECULAR CLONING, A LABORATORY MANUAL, Cold Springs Harbor Laboratory, New York (1989,1992), and in Ausubel et al, CURRENT PROTOCOLS IN MOLECULAR BIOLOGY, John Wiley and Sons, Baltimore, Md. (1989). Polymerase chain reaction (PCR) is carried out generally as in PCR PROTOCOLS: GUIDE TO METHODS AND APPLICATIONS, Academic Press, San Diego, Calif. (1990). Reactions and manipulations involving other nucleic acid techniques, unless stated otherwise, are performed as generally described in Sambrook, et al, 1989. MOLECULAR CLONING: A LABORATORY MANUAL, Cold Springs Harbor Laboratory Press, and methodology set forth in U.S. Pat. Nos. 4,666,828; 4,683,202; 4,801,531; 5,192,659; and 5, 2,057 and incorporated herein by reference. In-situ PCR in combination with Flow Cytometry can be used for detection of cells containing specific DNA and mRNA sequences (Testoni et al., Blood 87:3822 (1996)).

Standard methods in immunology known in the art and not specifically described are generally followed as in Stites et al. (eds), BASIC AND CLINICAL IMMUNOLOGY, 8th Ed., Appleton & Lange, Norwalk, CT (1994); and Mishell and Shigi (eds), SELECTED METHODS IN CELLULAR IMMUNOLOGY, W. H. Freeman and Co., New York (1980).

Immunoassays

In general, immunoassays are employed to assess a specimen such as for cell surface markers or the like. Immunocytochemical assays are well known to those skilled in the art. Both polyclonal and monoclonal antibodies can be used in the assays. Where appropriate other immunoassays, such as enzyme-linked immunosorbent assays (ELISAs) and radioimmunoassays (RIA), can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,2 2; 3,901,654; 3,935,074; 3,984,533; 3,996,345; 4,034,074; 4,098,876; 4,879,219; 5,011,771 and 5,281,521 as well as Sambrook et al., MOLECULAR CLONING: A LABORATORY MANUAL, Cold Springs Harbor, N.Y. (1989).

Gene Therapy

Gene therapy as used herein refers to the transfer of genetic material (e.g., DNA or RNA) of interest into a host to treat or prevent a genetic or acquired disease or condition. The genetic material of interest encodes a product (e.g., a protein, polypeptide, peptide, functional RNA, antisense) whose in vivo production is desired. For example, the genetic material of interest encodes a hormone, receptor, enzyme, polypeptide or peptide of therapeutic value. Alternatively, the genetic material of interest encodes a suicide gene. For a review see “Gene Therapy” in ADVANCES IN PHARMACOLOGY 40, Academic Press, San Diego, Calif., 1997.

Delivery of Cells

The cells of the present invention are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art.

In the method of the present invention, the cells of the present invention can be administered in various ways as would be appropriate to implant in the central nervous system, including but not limited to parenteral administration, intrathecal administration, intraventricular administration. and intranigral administration.

### EXAMPLES

Human Microalia Culture:

Brain tissue (mostly telencephalon) was obtained from 12-18 weeks gestation embryos. This use of human tissue was review and approved by the Ethics Committee of the University of British Columbia. The brain tissue was incubated in phosphate buffered saline (PBS) containing 0.25% trypsin and 40 ug/ml Dnase for 30 min at 37° C. and then dissociated into single cells by gentle pipetting. Dissociated single cells were grown in T75 flasks in feeding medium consisting of Dulbecco's modified Eagle's medium (IJMEM) to which the following were added: 5% horse serum, 5 mg/ml D-glucose and 20 ug/ml gentamicin. Following 2-3 weeks of growth in flasks, free-floating microglia were collected and plated in 6-well plates coated with poly-L-lysine. For the last part of this time, microglia were exposed to granulocyte macrophage colony stimulating factor (GM-CSF) at final concentration of 8 ug/ml for 9-12 days with medium change every 3 days. GM-CSF treatment stimulated proliferation of microglia resulting in a 3-4-fold increase in microglial population. The microglia isolated from primary cultures of embryonic human telencephalon brain cells were mostly round cell with filopodia surrounding the cell bodies or slender cells with several primary branches and were from 8-12 um in size. Microglia composed in excess of 98% of plated cells as determined using the specific cell type-specific markers, ricinus communis agglutinin-1 and CD11b.

Retrovirus-media Gene Transfer:

Microglia treated with GM-CSF for 9-12 days were subjected to retrovirally mediated transduction of v-myc and subsequent cloning. An amphotropic replication-incompetent retroviral vector encoding v-myc oncogene transcribed from mouse leukemia virus LTR plus neomycin-resistant gene transcribed from an internal SV40 early promoter, was used to infect human microglia and induce propagation of immortalized human microglia cell lines. This amphotropic vector, PASK, was generated using the ecotropic retroviral vector encoding v-myc (obtained from ATCC, Rockville. Md.) to infect the PA318 amphotropic packaging line.

Successful infectants were selected and expanded. Infection of human microglia in 6-well plates was performed by the following procedures: 2 ml of supernatant from the PA318 packaging line an 8 ug/ml polybrene was added to microglia in 6-well plates and incubated for 4 hr at 37° C.; the solution was replaced with fresh feeding medium; and infection was repeated 24 hr later. After 72 hr following the infection, infected cells were selected with G418. Individual clones were generated by limiting dilution and propagated further. Several G-418 resistant colonies were isolated, expanded and were named HM06. One of these HMO6 cell lines, HMO6.AI was investigated for its cellular and molecular characteristics.

Characterization HMO6.AI Human Microglial Cells

HM06.A1 cells expressed surface antigens specific for microglial/macrophage lineage cells such as CD11b, CD68, HLA-DR, HLA-ABC and ricinus commuriis agglutinin lectin (RCA) as determined by imrnunocytochemistry (Antibody source: DAKO Diagnostics Canada, Inc., Mississauga, Ontario).

When the cells were exposed for two hours to medium containing carbon particles at 37° C., all cells were loaded with dark particles indicating that the cells are capable of active phagocytosis.

Other HM 6.AI cells were harvested and subjected to reverse transcriptase polymerase chain reaction (RT-PCR) analysis. Sequences for each cDNA were obtained from Genbank, and appropriate oligos were prepared. The HMO6.A1 cells expressed messages specific for microglia such as CD 11b, CD68 and B7-2 but did not express messages for GFAP (for astrocytes), MB [symbol] (for oligodendrocytes) or neurofllament protein-L (for nerve cells), In addition, HM06.A1 expressed messages for cytokines IL-1b, IL-6, 1L-8, IL-I 2, IL-15, TGF-b and chemokines MIP-1a, MIP-1b and MCP-1. All the cytokines and chemokines determined in H06.AI as shown above are specific and relevant cytokines released by activated human microglia in vivo and in vitro. The message for TNF-a, a cytotoxic proinflammatory cytokine involved in pathogenesis in various neurodegenerative diseases, was not detected. In addition, messages for ATP receptors and purinoreceptors P2Y1R and P2Y2R were also found in HMO6.AI cells, as expected from normal human microglia preparations.

Results obtained by immunohistochemistry and RT-PCR analyses on HMO6.A1 cells indicate that the cells possess phenotypes specifically and exclusively expressed by human microglia arid that HMO6.A1 cells are indeed a stable established cell line of human microglia.

In addition, Fura-2 fluorescence imaging has indicated that HMO6 cells response to platelet activating factor (PAF) and ATP with increased CA²⁺ influx. Upon stimulation of the HMO6 cells with interferon-y. they express B7-2 (CD82). a cell type-specific marker for antigen-presenting cells. In addition, HM06 cells are immunoreactive for HLA-ABC, HLA-DR and RCA-1 lectin, further supporting that the HM06 phenotype is specific for macrophage/ microglia type cells. HM06 cells also express mRNA for the chemokine receptor CXCR4.

### Table 1

### PHENOTYPES EXPRESSED BY HM06.AI HUMAN MICROGLIA LINE IMMUNOHISTOCHEMISTRY

| | |
|---|---|
| IMMUNOHISTOCHEMISTRY | |
| CD11b(Mac1) | + |
| CD6S | + |
| HLA-ABC | + |
| HLA-DR | + |
| RT-PCR ANALYSIS | |
| IL-1b | + |
| IL-6 | + |
| IL-8 | + |
| IL-12 | + |
| IL-15 | + |
| TGF-b | + |
| TNF-a | − |
| MIP-1a | + |
| MIP-1b | + |
| MCP-1 | + |
| P2Y1R | + |
| P2Y2R | + |

1. An immortalized human cell line with the characteristics of human microglia.

2. An immortalized human microglial cell line expressing the CD68 and CD11c antigens.

3. The immortalized human cell line of claim 1, wherein the cells have at least three of the following attributes: CD11b (Mac1), CD68, HLA-ABC, HLA-DR, IL-1b, IL-6, IL-8, IL-12, IL-15, TGF-b, TNF-a, MIP-Ia, MIP-1b, MCP-1, P21R, P2Y2R.

4. A method of transforming human microglial cells into an immortalized cell line the method comprising
a. obtaining human microglial cells;
b. culturing human microglial cells;
c. transfecting the human microglial cells with amphotropic replication-incompetent retroviral vector encoding v-myc oncogene transcribed from mouse leukemia virus LTR plus neomycin-resistant gene transcribed from an internal SV40 early promoter; and
d. expanding transfectants.

5. A method of testing drugs for effects on human microglial cells, the method comprising
a. providing immortalized human microglial cells;
b. exposing the cells to the drug for a sufficient time to permit a reaction; and
c. observing the reaction of the cells.

6. A method of treating an individual experiencing a neurodegenerative disorder, comprising administering a sufficient quantity of the immortalized human microglia cells to the individual with the neurodegenerative disorder.

7. The method of claim 6, wherein the neurodegenerative disorder involves the individual's microglia cells.

8. The method of claim 6, wherein the neurodegeneration disorder is Parkinson's Disease, Alzheimer's diseases ischemia, spinal cord damage, ataxia, or alcoholism.
